(19) United States
(12) Patent Application Publication
(10) Pub. No.: US 2013/0085325 A1
(43) Pub. Date: Apr. 4, 2013

(54) HAND-MOUNTED, VIDEO-GUIDED SYSTEM FOR TREATING PERITONITIS AND OTHER MEDICAL CONDITIONS

(71) Applicants: Bradbury Fuller, Weybridge, VT (US); Eileen Fuller, Weybridge, VT (US); Robert Degon, Bellingham, MA (US); Paul DiCarlo, Middleboro, MA (US); Jason L. Hamilton, Dartmouth, MA (US); Harold M. Martins, Newton, MA (US); Eric Meade, Slatersville, RI (US); Amanda Mitchell, Worcester, MA (US); Blake Sama, Boston, MA (US); Allison Waller, Attleboro, MA (US); Anthony Wong, Franklin, MA (US)

(72) Inventors: Bradbury Fuller, Weybridge, VT (US); Eileen Fuller, Weybridge, VT (US); Robert Degon, Bellingham, MA (US); Paul DiCarlo, Middleboro, MA (US); Jason L. Hamilton, Dartmouth, MA (US); Harold M. Martins, Newton, MA (US); Eric Meade, Slatersville, RI (US); Amanda Mitchell, Worcester, MA (US); Blake Sama, Boston, MA (US); Allison Waller, Attleboro, MA (US); Anthony Wong, Franklin, MA (US)

(21) Appl. No.: 13/592,083

(22) Filed: Aug. 22, 2012

Related U.S. Application Data
(60) Provisional application No. 61/575,485, filed on Aug. 22, 2011.

(51) Int. Cl. A61B 1/00; A61M 1/00; A61M 3/02

(52) U.S. Cl. CPC A61B 1/00147; A61M 3/0283; A61M 1/0039
USPC 600/102

(57) ABSTRACT

A hand-mounted, video-guided system comprising:
an element for mounting to at least one finger of a user;
a video input carried by said element;
an irrigation line carried by said element;
a fenestrated suction head for mounting to the hand of a user, said fenestrated suction head comprising a hollow body having fenestrations formed therein, and a suction line having a fenestrated distal end disposed within the interior of said hollow body.

### REFERENCE TO PENDING PRIOR PATENT APPLICATION

This patent application claims benefit of pending prior U.S. Provisional Patent Application Ser. No. 61/575,485, filed Aug. 22, 2011 by Bradbury Fuller for HAND MOUNTED VIDEO GUIDED PERITONEAL LAVAGE SYSTEM AND METHOD OF USE (Attorney's Docket No. FULLER-01 PROV), which patent application is hereby incorporated herein by reference.

### FIELD OF THE INVENTION

This invention relates to medical procedures and apparatus in general, and more particularly to methods and apparatus for treating peritonitis. This invention also has other medical and non-medical applications.

### BACKGROUND OF THE INVENTION

Peritonitis is an inflammation of the peritoneum, the thin layer of tissue which lines the inner wall of the abdomen and encompasses most of the abdominal organs. Typically the primary symptom of peritonitis is that the abdomen will be painful or tender. This pain or tenderness may become more pronounced when the abdomen is touched or when the person moves, and the abdomen may look or feel bloated.

Peritonitis is surprisingly common, even in the developed countries, and can be fatal to those who are affected by it. When properly and timely treated, peritonitis has a mortality rate of less than 10%, however, the mortality rate rises to about 40% among the elderly. Untreated peritonitis is almost always fatal.

Perforation of the gastrointestinal tract is a common cause of peritonitis. More particularly, perforation of the gastrointestinal tract can result in a life-threatening bacterial infection involving the peritoneal cavity, i.e., bacterial peritonitis. Among other things, perforation of the gastrointestinal tract allows infected fluid, proliferating with microorganisms, to enter the peritoneal cavity from the gastrointestinal tract. This infected fluid provokes an inflammatory response on the involved surfaces of the peritoneal cavity. The inflammatory response produces proteins (i.e., cytokines) that act in concert to recruit cells (i.e., fibroblasts) to the areas of contamination so as to (i) produce antibodies to assist in the eradication of microorganisms, (ii) deposit proteinaceous material (i.e., fibrin) which acts as a biologic sealant to physically seal off and isolate the source of contamination, and (iii) eliminate (through the process of phagocytosis) injured and necrotic tissues. The process of wound healing, with the production of collagen leading to scar formation, proceeds as well. However, as the degree of contamination and the extent of the infection increases, the inflammatory response can result in widespread and deleterious activation of these same processes elsewhere in the abdomen, as well as in other organ systems within the body, ultimately leading to failure of multiple organ systems and death. Widespread scar formation can also lead to excessive adhesion formation, potentially leading to subsequent adhesive bowel obstructions and complicating future abdominal procedures.

A laparotomy (i.e., a surgical procedure involving a large incision through the abdominal wall so as to gain access to the abdominal cavity, and also sometimes known as a coeliotomy) is a typical surgical treatment for bacterial peritonitis. The laparotomy involves, first and foremost, control of the source of the infection and removal/evacuation of the contaminated products of the inflammatory response, i.e., surgical removal of the perforated segment of bowel or closure of the site of the perforation, coupled with the evacuation of all infected fluid and a thorough lavage and debridement of the contaminated surfaces within the abdominal cavity. Conventional practice typically involves the manual pouring of isotonic, sterile salt solutions, sometimes also containing antimicrobial medication in solution, into the peritoneal cavity, coupled with suction evacuation of the fluid from the cavity, so as to reduce the bacterial load within the abdominal cavity. The goal is to reduce the bacterial load within the abdominal cavity to a level that can then be eradicated by a combination of systemic antibiotic therapy (administered intravenously) and the inflammatory response generated by the body's immune system, thereby restoring sterility to the abdominal cavity.

The degree to which mechanical removal of all evidence of infected material, along with the products of the immune response, can be maximized, potentially renders the immune response less critical to recovery and, due to the possible dilution and/or removal of regulatory factors, also has the potential for down regulating the intensity of the immune response, thereby lowering the potential for the more deleterious effects that the immune response can have on the abdominal cavity as well as the other organ systems in the body. Unfortunately, current treatment approaches (both methods and apparatus) are insufficient to consistently and adequately remove the infected material and immune response products from the abdominal cavity. As a result, even with surgical intervention, peritonitis may persist, often with fatal results. In this respect it should be appreciated that generalized peritonitis is associated with a high mortality rate in the most serious cases. Despite great improvements in the standards of intensive care, the morbidity and mortality associated with this condition have not significantly improved, in part due to recurrent intra-abdominal sepsis which is often associated with the difficulties in consistently and adequately accessing, with abdominal lavage and debridement, the loci of infected tissue or residual gastrointestinal tract content commonly not reached when located between or behind the organs within the abdominal cavity.

It is fairly well recognized that an aggressive policy of repeated serial abdominal lavage and debridement may be the only treatment that offers an improvement in the morbidity and mortality from generalized peritonitis. To this end, many doctors in the 1980s began to practice routine repeat laparotomy, in which a second operation is automatically scheduled a fixed period of time after the first laparotomy. This approach enables the surgeon, during the second laparotomy, to identify any re-growth of the infection and remove it. However, repeated laparotomies are costly, and such major surgery can have serious negative outcomes with patients as their bodies are further stressed by the repeated procedures.

While such prior art approaches have benefits, they also suffer from significant drawbacks. Significantly, due to the inability of current approaches to locate, identify, adequately lavage and debride infected tissue, etc., patients too often must suffer from the stress and risk of a second or even a third laparotomy.

Thus there is a need for a new approach which would permit a surgeon to more effectively locate, identify, adequately lavage and debride infected tissue within the peritoneal cavity, whereby to better treat peritonitis. Among other things, there is a need for a new approach which would permit a surgeon to directly observe, irrigate and debride infected tissue within the peritoneal cavity, whereby to better treat peritonitis.

### SUMMARY OF THE INVENTION

The present invention provides a new approach which permits a surgeon to more effectively locate, identify, adequately lavage and debride infected tissue within the peritoneal cavity, whereby to better treat peritonitis. Among other things, the present invention provides a new approach which permits a surgeon to directly observe, irrigate and debride infected tissue within the peritoneal cavity, whereby to better treat peritonitis.

In one form of the present invention, there is provided a hand-mounted, video-guided system comprising:

an element for mounting to at least one finger of a user;

a video input carried by said element;

an irrigation line carried by said element;

a fenestrated suction head for mounting to the hand of a user, said fenestrated suction head comprising a hollow body having fenestrations formed therein, and a suction line having a fenestrated distal end disposed within the interior of said hollow body.

In another form of the present invention, there is provided a method for treating a patient, comprising:

providing a hand-mounted, video-guided system comprising:
an element for mounting to at least one finger of a user;
a video input carried by said element;
an irrigation line carried by said element;
a fenestrated suction head for mounting to the hand of a user, said fenestrated suction head comprising a hollow body having fenestrations formed therein, and a suction line having a fenestrated distal end disposed within the interior of said hollow body;

mounting said apparatus to the hand of a user;

advancing the hand of a user into a body cavity; and

providing lavage therapy to the patient using said irrigation line and said fenestrated suction head while visualizing the anatomy using said video input.

In another form of the present invention, there is provided apparatus comprising:

an element for mounting to at least one finger of a user; and

a video input carried by said element.

In another form of the present invention, there is provided a method for examining or treating a patient, comprising:

providing apparatus comprising:
an element for mounting to at least one finger of a user; and
a video input carried by said element;

mounting said apparatus to the hand of a user;

advancing the hand of a user into a body opening or cavity; and

visualizing the anatomy using said video input.

In another form of the present invention, there is provided a method for viewing a confined space and/or performing a task in the confined space, comprising:

providing apparatus comprising:
an element for mounting to at least one finger of a user; and
a video input carried by said element;

mounting said apparatus to the hand of a user;

advancing the hand of a user into a confined space; and

viewing the confined space using the video input.

In another form of the present invention, there is provided a hand-mounted video system comprising:

a video input for acquiring video data; and

means for mounting said video input to the hand of a user.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIGS. 1-5 are schematic views showing a hand-mounted, video-guided system formed in accordance with the present invention;

FIGS. 6-16, 16A and 17-21 are schematic views showing additional constructions for the hand-mounted, video-guided system of the present invention;

FIGS. 22-25 are schematic views showing user interface controls which may be provided for the hand-mounted, video-guided system of the present invention;

FIGS. 26-32 are schematic views showing an exemplary construction for the hand-mounted, video-guided system of the present invention;

FIGS. 33-39 are schematic views showing another exemplary construction for the hand-mounted, video-guided system of the present invention;

FIGS. 40-45 are schematic views showing still another exemplary construction for the hand-mounted, video-guided system of the present invention;

FIG. 46 is a schematic view showing another form of the present invention;

FIG. 47 is a schematic view showing still another form of the present invention; and

FIG. 48 is a schematic view showing yet another form of the present invention.

### DETAILED DESCRIPTION OF THE PREFERRED EMBODIMENTS

### Peritoneal Lavage System

The present invention provides a new approach which permits a surgeon to more effectively locate, identify, adequately lavage and debride infected tissue within the peritoneal cavity, whereby to better treat peritonitis. Among other things, the present invention provides a new approach which permits a surgeon to directly observe, irrigate and debride infected tissue within the peritoneal cavity, whereby to better treat peritonitis.

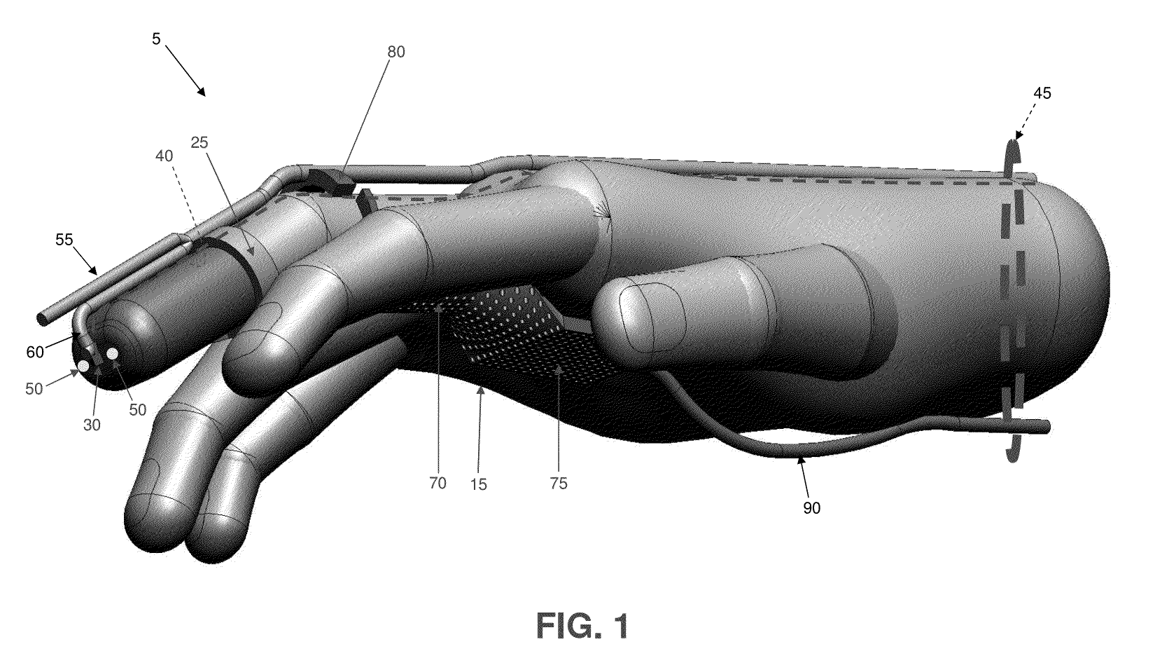

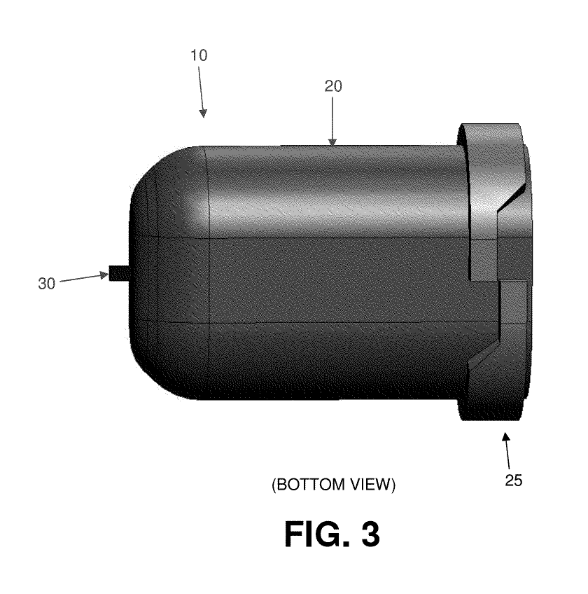

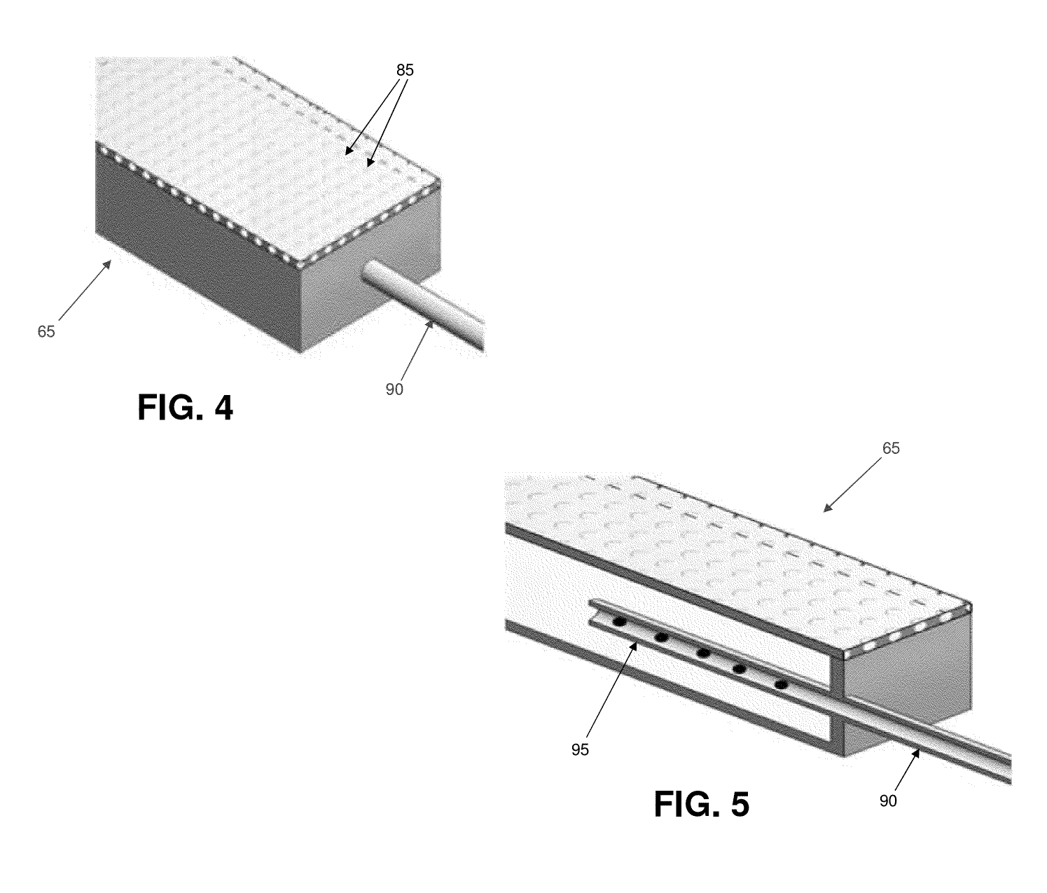

More particularly, and looking first at FIGS. 1-5, there is shown a novel hand-mounted, video-guided system 5 formed in accordance with the present invention. Hand-mounted, video-guided system 5 generally comprises a finger cot 10 (FIGS. 1-3) for providing illumination, visualization and irrigation at the tip of a finger, and a fenestrated suction head 15 (FIGS. 1, 2, 4 and 5) for providing suction at the base of the finger and/or at the palm of the hand. Hand-mounted, video-guided system 5 allows a user to reach their hand into a remote site in the peritoneal cavity, thereby providing illumination and visualization of the remote site accessed by the hand. As this occurs, hand-mounted, video-guided system 5 preserves the user's normal tactile sensation from their fingers, thereby facilitating finger fracture dissection and debridement of loculated infected fluid collections. Hand-mounted, video-guided system 5 also provides lavage (irrigation and suction) at the remote site accessed by the hand. In this way, hand-mounted, video-guided system 5 provides a new and improved way to treat peritonitis within the peritoneal cavity.

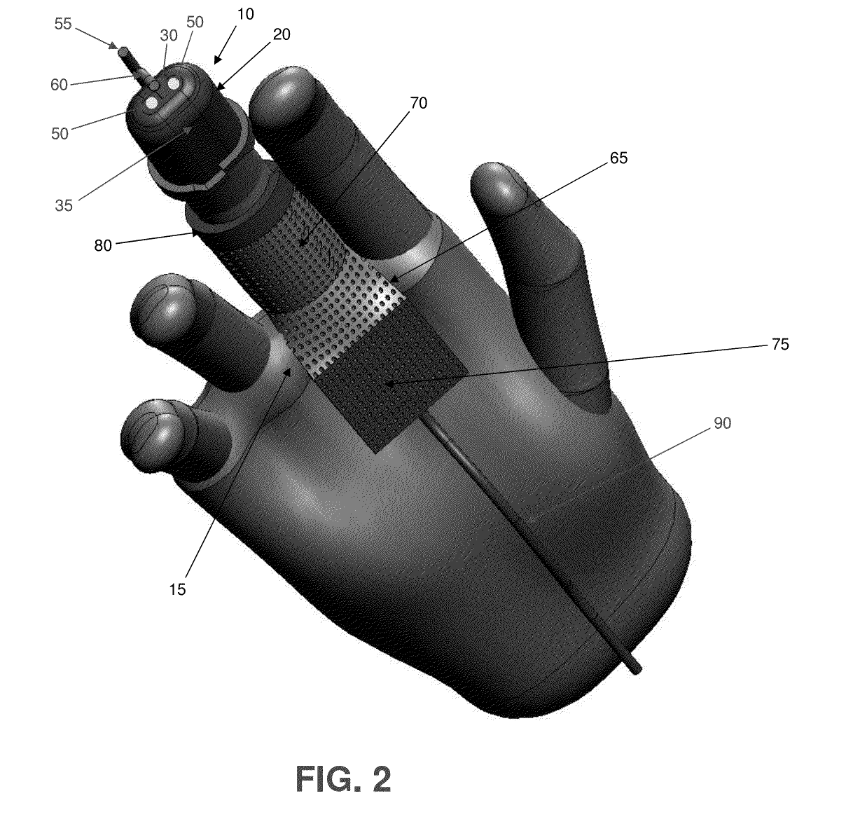

More particularly, finger cot 10 comprises a sleeve 20 sized to fit over a finger of the user. Finger cot 10 is formed out of a thin, flexible material (e.g., standard medical grade polymer such as acrylonitrile butadiene styrene, PEEK, polyvinyl chloride, polysulfone, polyamide, polyimide, etc.) such that the user's tactile sensation is retained through the finger cot. Finger cot 10 is preferably releasably secured to a finger of the user (e.g., to the middle finger of the user, as shown in FIGS. 1 and 2) via a releasable locking ring 25 formed at the proximal end of finger cot 10. In this form of the invention, the releasable locking ring 25 is constructed so that it can be appropriately closed down around the finger of a user so as to size the releasable locking ring to the size of the finger and thereby secure the releasable locking ring (and hence finger cot 10) to the finger of the user.

Alternatively, other methods and apparatus can be used to releasably secure finger cot 10 to a finger of the user, e.g., a “sizable” ring or clip (for example, a bendable or malleable ring or clip), an elastomeric band, etc.

Alternatively, if desired, finger cot 10 may comprise a portion of a glove which covers the hand of the user.

Finger cot 10 carries a video input 30 at the distal end of the finger cot, with the video input being spaced from the pad 35 of the finger so that video input 30 does not interfere with the user's tactile sense from the pad of the finger. The visual data acquired by video input 30 is transmitted along the length of finger cot 10, and then across the back of the hand, via a transmission pathway 40. If desired, an elastic cuff 45 may be used to hold transmission pathway 40 to the wrist of the user so as to keep the transmission pathway from interfering with use of the user's hand.

Transmission pathway 40 is configured to be connected to appropriate visualization elements (not shown) of the sort well known in the art (e.g., in the art of endoscopes) so that the visual data captured by video input 30 can be presented to the user for viewing and/or so that the visual data captured by video input 30 can be recorded (e.g., for later viewing). By way of example but not limitation, such visualization elements may comprise a video display, etc.

In one preferred form of the invention, video input 30 comprises a video chip (e.g., a CCD chip, a CMOS chip, etc.) and appropriate lens(es) for acquiring an image of the region immediately in front of the finger carrying finger cot 10, and transmission pathway 40 comprises wires for transmitting the acquired image data to appropriate visualization elements (e.g., a video display, etc.). In another preferred form of the invention, video input 30 comprises an appropriate lens or lenses, and transmission pathway 40 comprises a fiberoptic cable for transmitting the acquired image data to appropriate visualization elements (e.g., a video camera, a video display, etc.). This latter form of the invention can be advantageous in that it can reduce the amount of heat generated at the tip of finger cot 10.

One or more illumination elements 50, preferably in the form of one or more illumination LEDs or one or more light fibers, are provided on finger cot 10 adjacent to video input 30 so as illuminate the region which is monitored by video input 30. As a result, video input 30 is able to acquire images of a patient's anatomy even when the video input is disposed in an area lacking ambient lighting (e.g., in a remote corner of the peritoneal cavity). Illumination elements 50 are preferably provided on the distal end of finger cot 10, spaced from the pad 35 of the finger, so that the illumination elements do not interfere with the user's tactile sense from the pad of the finger. In one preferred form of the present invention, illumination elements 50 comprise two illumination LEDs with supporting electrical components (e.g., a remote electrical power source, wires to connect the remote electrical power source to the LEDs, etc.). In another preferred form of the present invention, illumination elements 50 comprise two light fibers with supporting light-generating components (e.g., a remote light source and remote electrical components for energizing the remote light source).

As a result of the foregoing construction, when a finger cot 10 is disposed on a finger of a user, the user can use the video input 30 carried by the finger cot to visualize the region in front of their finger, thereby allowing the user to visualize regions which can be accessed by their hand but which may be otherwise unavailable to direct viewing by the user. Significantly, this may be done while retaining the tactile feedback normally provided by the user's hand.

Finger cot 10 also carries an irrigation line 55 for irrigating the region in front of finger cot 10. Irrigation line 55 is preferably mounted to the dorsal portion of finger cot 10 so that the irrigation line does not interfere with the tactile sense of the user from the pad of the finger. Alternatively, irrigation line 55 may be mounted to a side portion of finger cot 10 so that the irrigation line does not interfere with the tactile sense of the user from the pad of the finger. Irrigation line 55 preferably extends across the back of the user's hand and, if desired, elastic cuff 45 may be used to hold irrigation line 55 to the wrist of the user so as to keep the irrigation line from interfering with use of the user's hand. In one preferred form of the invention, transmission pathway 40 may be mounted to irrigation line 55 so as to provide a more streamlined construction for the system and to reduce the possibility of snagging on tissue, etc.

If desired, a supplemental irrigation line 60 may also be provided for irrigating the lens(es) of video input 30. Supplemental irrigation line 60 preferably also extends across the back of the user's finger and hand and, if desired, elastic cuff 45 may be used to hold supplemental irrigation line 60 to the wrist of the user so as to keep the supplemental irrigation line from interfering with use of the user's hand. Alternatively, if desired, supplemental irrigation line 60 may be mounted to a side portion of finger cot 10 and then extend across the back of the user's hand. In one preferred form of the invention, supplemental irrigation line 60 may be mounted to irrigation line 55 so as to provide a more streamlined construction for the system and to reduce the possibility of snagging on tissue, etc.

Hand-mounted, video-guided system 5 also comprises a fenestrated suction head 15 (FIGS. 1, 2, 4 and 5) for providing suction at the base of the finger and/or the palm of the hand. More particularly, fenestrated suction head 15 generally comprises a hollow body 65 for mounting to a finger of the user and/or the palm of the user's hand. By way of example but not limitation, in one preferred form of the invention, hollow body 65 of fenestrated suction head 15 comprises a curved hollow shell 70 configured to seat against the underside of a user's finger at the base of the finger, and a hollow palm extension 75 configured to seat against the palm of the hand at the base of the finger, with the interior of curved hollow shell 70 communicating with the interior of hollow palm extension 75. By way of further example but not limitation, curved hollow shell 70 may be mounted to a finger of the user via a strap 80, with strap 80 serving to hold both curved hollow shell 70 and hollow palm extension 75 in place on the hand of the user. Alternatively, strap 80 may be replaced by a “sizable” ring or clip, or elastomeric band, etc. In one preferred form of the invention, strap 80 may also be attached to irrigation line 55 and, if desired, transmission pathway 40 and supplemental irrigation line 60, so as to provide additional support for these elements as they extend along the back of the finger. Preferably such a connection between strap 80, irrigation line 55, transmission pathway 40 and supplemental irrigation line 60 is adjustable so as to accommodate different hand sizes. Fenestrations 85 are formed in the outer surface of fenestrated suction head 15.

A suction line 90 has its fenestrated distal end 95 disposed within the interior of hollow body 65 of fenestrated suction head 15 and extends across the palm of the hand for connection to a source of suction. If desired, elastic cuff 45 may be used to hold suction line 90 to the wrist of the user so as to keep the suction line from interfering with movement of the user's hand. Binding suction line 90 to the wrist of the user with elastic cuff 45 may also help to hold fenestrated suction head 15 in place on the hand of the user.

As a result of this construction, suction line 90 may be used to establish suction in hollow body 65 of fenestrated suction head 15 (e.g., in curved hollow shell 70 and in hollow palm extension 75), so that fluid in the vicinity of fenestrated suction head 15 is drawn into the interior of the fenestrated suction head 15 via fenestrations 85 for evacuation via suction line 90. Thus it will be appreciated that fluid in the region adjacent to the base of the finger and/or the palm of the hand can be drained by the suction provided to fenestrated suction head 15.

As a result of the foregoing construction, a user can mount finger cot 10 to the tip of a finger, and can mount fenestrated suction head 15 to the base of the finger and/or the palm of the hand. The user can then advance their hand into the peritoneal cavity to explore for infection. As the user explores the peritoneal cavity for infection, they can manipulate the tissue, with their tactile sense intact. Furthermore, the user is able to visualize the region in front of their hand, using the video input 30 located on finger cot 10, with illumination elements 50 providing appropriate illumination for video input 30, to ensure the clearing of turbid contaminated fluid and fibrinous exudate. When it is desired to irrigate tissue, irrigation is provided at the tip of the hand via irrigation line 55, and when it is desired to suction out fluid, fenestrated suction head 15 provides suction to the area. Thus it will be seen that hand-mounted, video-guided system 5 comprises a lavage system which is carried into the peritoneal cavity on the hand of the user and integrated into the natural hand movements of the user as the user explores for, and treats, peritonitis while under direct visualization.

### Additional Constructions

It should be appreciated that various modifications may be made to the hand-mounted, video-guided system 5 without departing from the scope of the present invention.

By way of example but not limitation, where finger cot 10 comprises a portion of a glove which covers the hand of the user, fenestrated suction head 15 may be incorporated into the construction of that glove, i.e., fenestrated suction head 15 may be integrated into that glove. As a result, when the user puts on the glove, the user simultaneously mounts video input 30, illumination elements 50, irrigation line 55, supplemental irrigation line 60 and fenestrated suction head 15 to their hand in one motion. In this form of the invention, transmission pathway 40 and other supporting elements of the system are preferably also mounted to the glove.

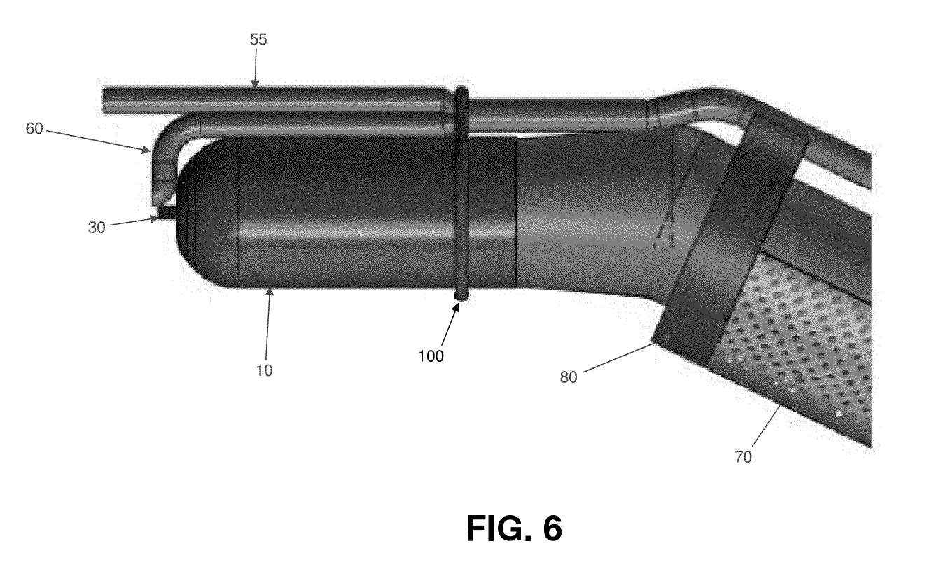

By way of further example but not limitation, releasable locking ring 25, which is used to releasably hold finger cot 10 to the finger of a user, may be replaced by an alternative structure, e.g., by a “sizable” ring or clip (for example, a bendable or malleable ring or clip), or by an elastomeric band 100 such as is shown in FIG. 6, or by other fixation devices compatible with the present invention.

Furthermore, and looking now at FIG. 7, if desired, a blunt hood 105 may be provided at the tip of finger cot 10. Blunt hood 105 can help protect video input 30 and/or illumination elements 50 from damage in the event that they should encounter a hard object (e.g., a surgical instrument). Blunt hood 105 can also help keep video input 30 and/or illumination elements 50 clear of fluids and foreign matter.

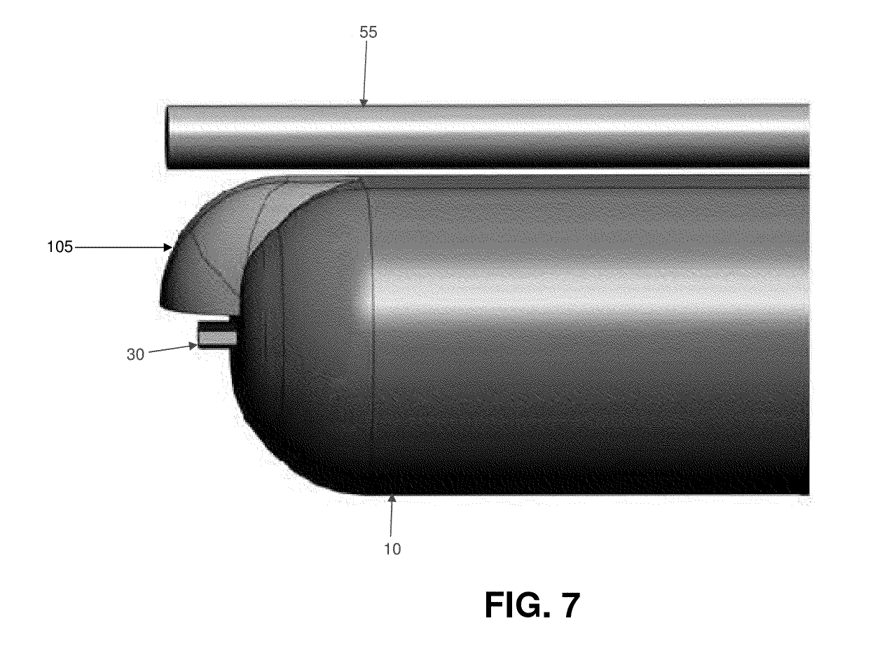

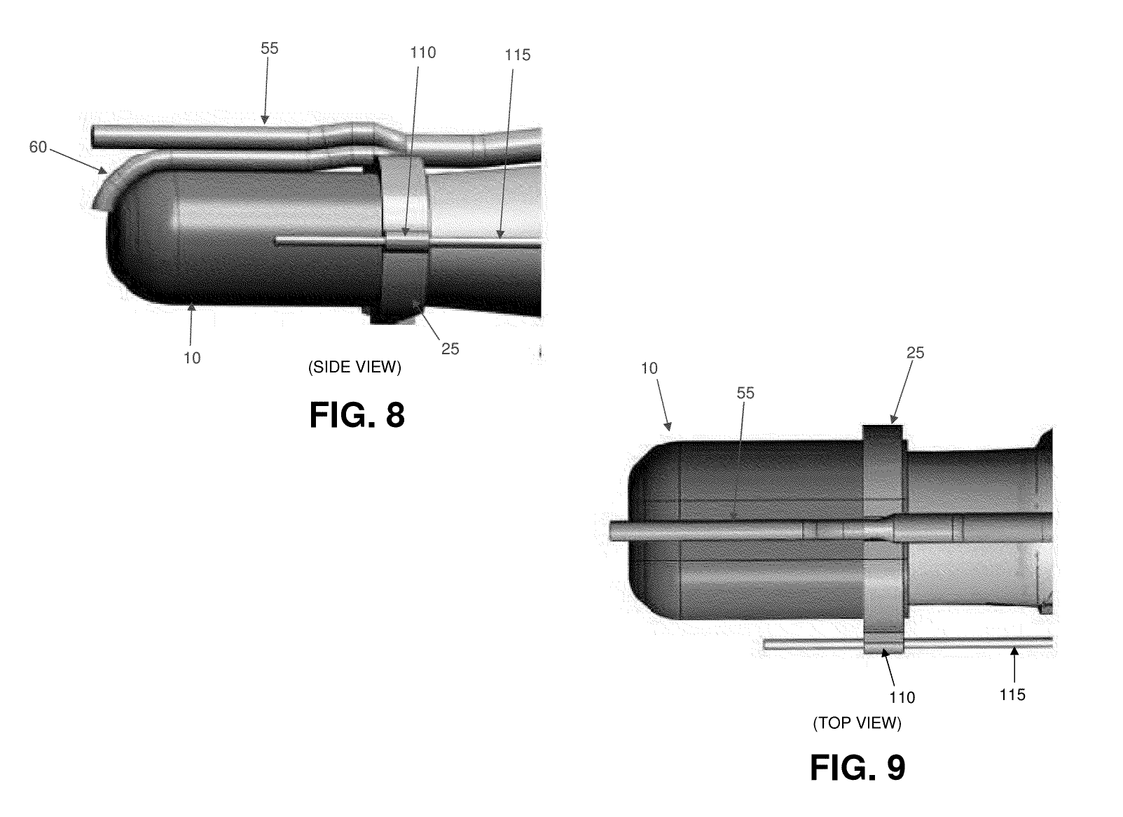

In another form of the invention, and looking now at FIGS. 8 and 9, releasable locking ring 25 can be provided with a mount 110 for slidably receiving instrumentation 115 (e.g., a biopsy instrument, a cauterizing device, a suction device, a laser, an ultrasound imager, an endoscope, an ablative device, a Blue light or fluorescent light, a balloon device for expansion, compression or occlusion, a tourniquet or clip device for compression or occlusion, a device for deployment of hemostatic agents to seal bleeding vessels, etc.), whereby the instrumentation can be delivered to the surgical site by the hand of the user and then, when needed, can be advanced distally so as to access the desired tissue (and can be retracted proximally when it is no longer needed). Thus, mount 110 allows instrumentation 115 to be advanced or retroacted, telescopically, relative to mount 110 (and hence relative to the finger of the user). In one preferred form of the invention, releasable locking ring 25 and mount 110 are configured so as to hold instrumentation 115 adjacent to the side of the user's finger.

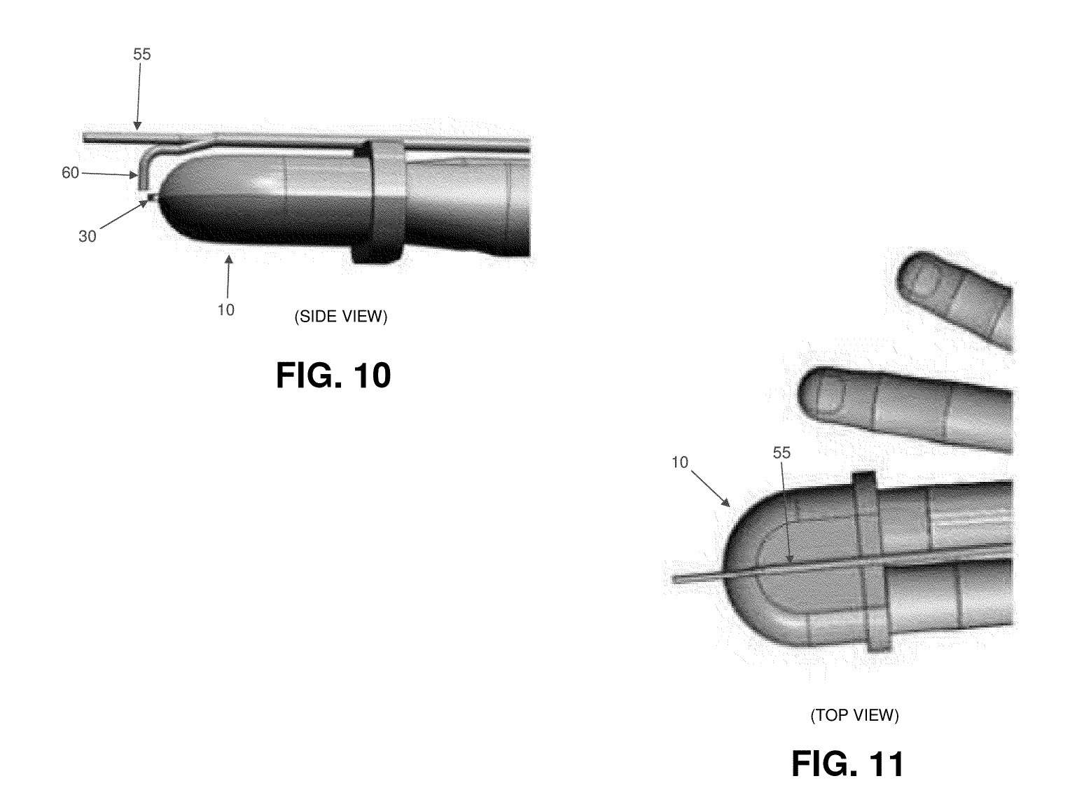

In some circumstances, there may be an advantage in carrying finger cot 10 to the surgical site on more than one finger, e.g., so as to increase stability, combine function over a greater area, etc. To that end, if desired, finger cot 10 may be sized to receive two or more fingers of the user. See, for example, FIGS. 10 and 11.

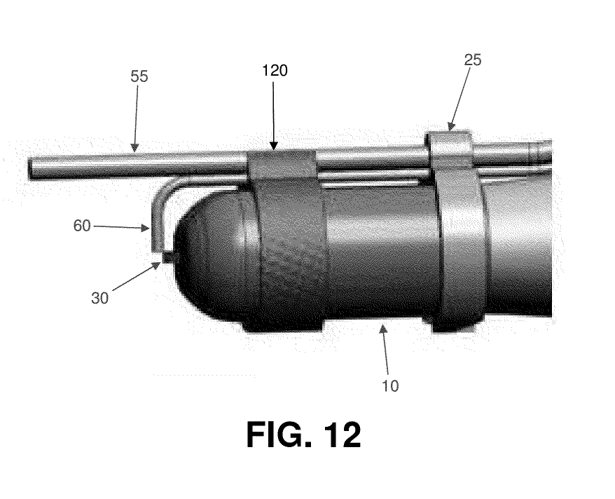

It is also anticipated that, in some circumstances, it can be advantageous to provide extendable irrigation for hand-mounted, video-guided system 5. To this end, and looking now at FIG. 12, irrigation line 55 may be slidably mounted to finger cot 10 so as to allow irrigation line 55 to telescope forward when desired (and to retract proximally when desired). In this form of the invention, additional stability may be provided for the telescoping irrigation line 55 via a supplemental sliding mount 120.

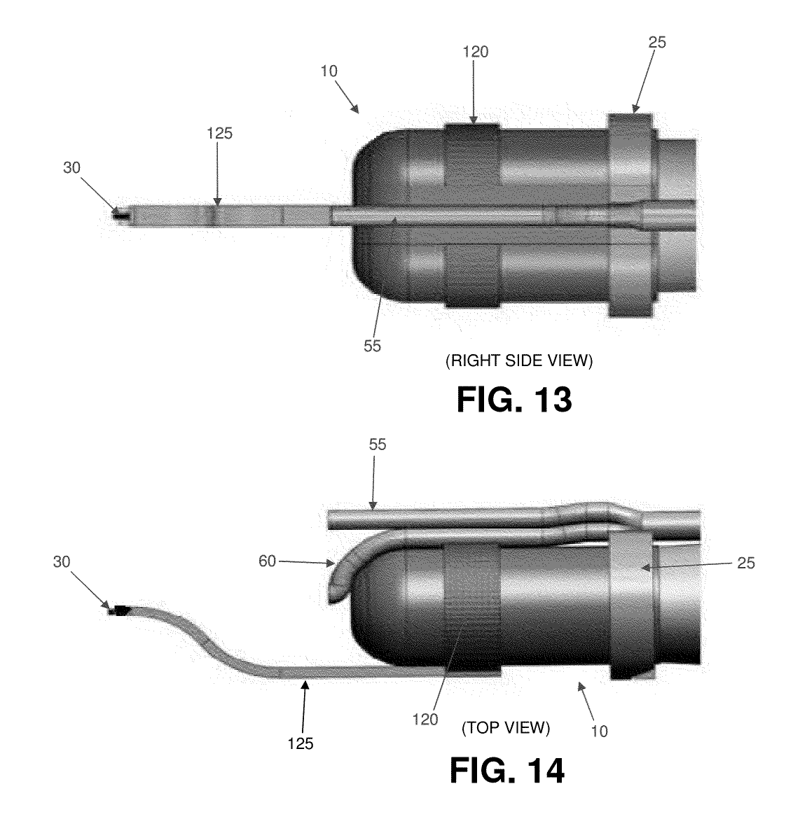

Furthermore, in some circumstances, it can be advantageous to provide an extendable video input 30 for hand-mounted, video-guided system 5. Accordingly, and looking now at FIGS. 13 and 14, in another preferred form of the invention, video input 30 (and preferably illumination elements 50 as well) may be mounted to a support 125 which is itself slidably mounted to finger cot 10 (e.g., via supplemental mount 120) so as to allow video input 30 to telescope forward (and to retract proximally) when desired.

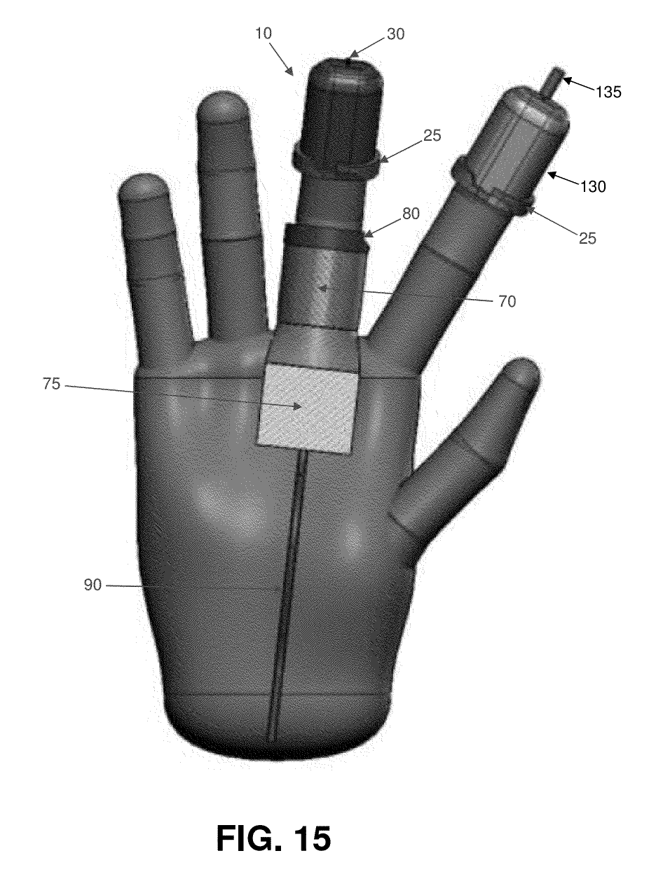

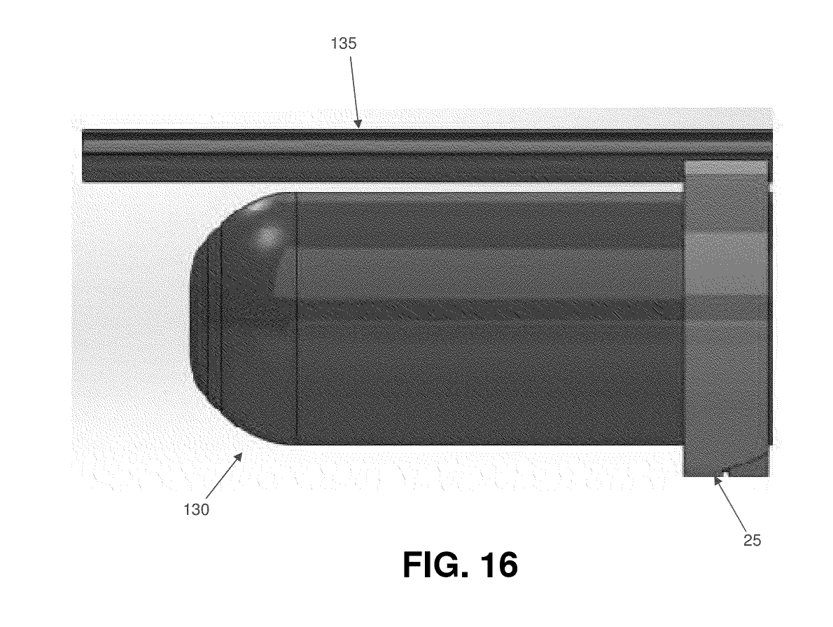

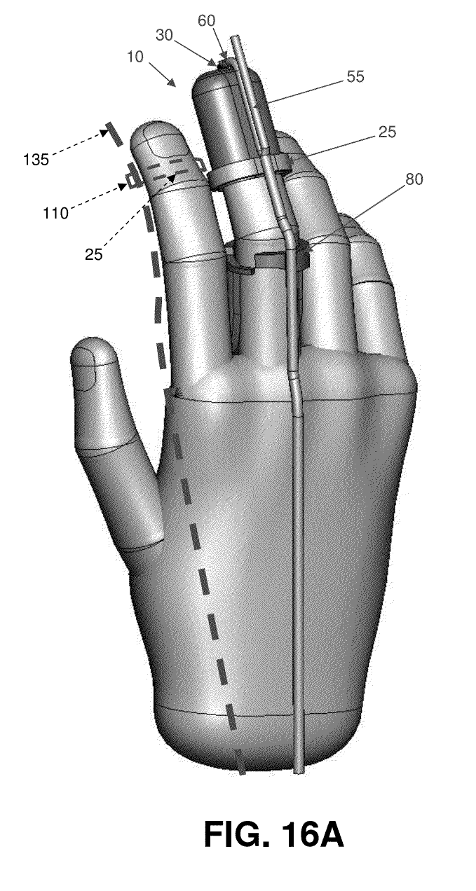

It is also possible for other fingers to carry auxiliary devices thereon. By way of example but not limitation, and looking now at FIGS. 15 and 16, where one finger carries the aforementioned finger cot 10 and the aforementioned fenestrated suction head 15, another finger may carry an auxiliary finger cot 130 carrying an auxiliary device 135 (e.g., an electrocautery device, a suction device, a laser, a biopsy instrument, an ultrasound imager, an endoscope, an ablative device, a Blue light or fluorescent light, a balloon device for expansion, compression or occlusion, a tourniquet or clip device for compression or occlusion, a device for deployment of hemostatic agents to seal bleeding vessels, etc.) thereon. Alternatively, an additional finger mount may be provided for positioning on a finger other than the finger carrying the aforementioned finger cot 10, with this additional finger mount being configured to telescopically support an auxiliary device 135. By way of example but not limitation, and looking now at FIG. 16A, an additional locking ring 25, carrying a mount 110 for slidably receiving auxiliary device 135, can be provided for another finger of a user, whereby to telescopically support auxiliary device 135. It will be appreciated that this additional finger mount may also comprise various other constructions consistent with the present invention, e.g., the additional finger mount may comprise a “sizable” ring or clip (for example, a bendable or malleable ring or clip, an elastomeric band, etc.) having a mount for telescopically carrying an auxiliary device.

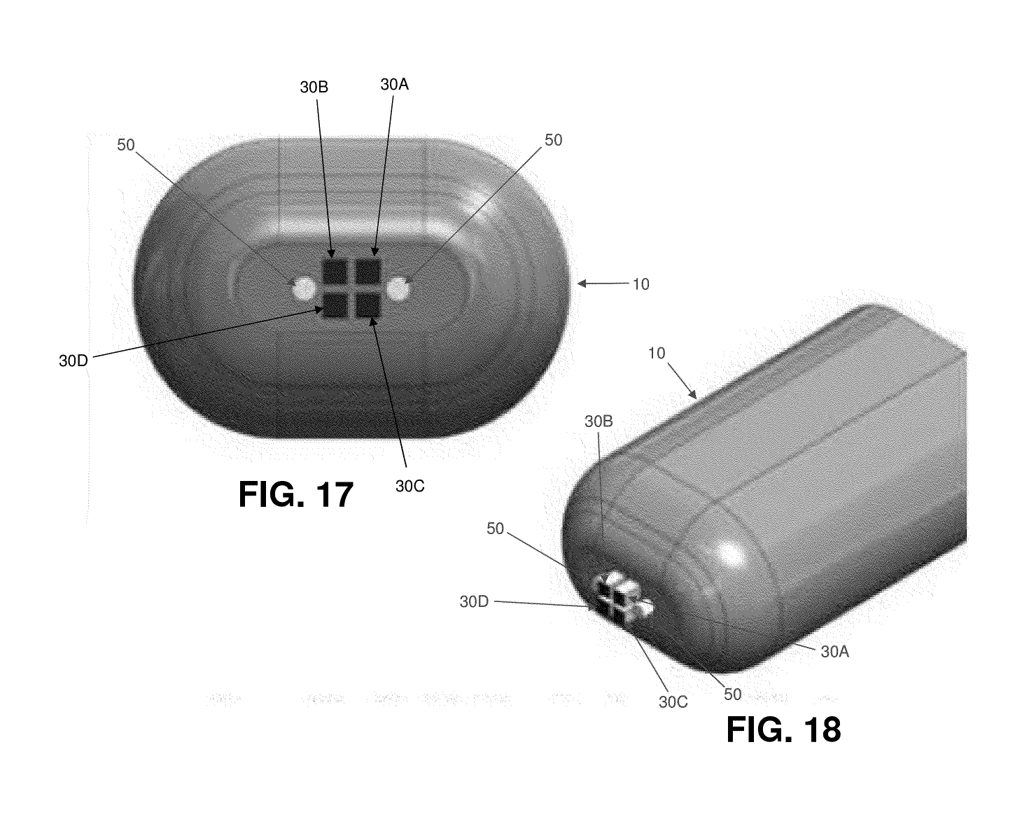

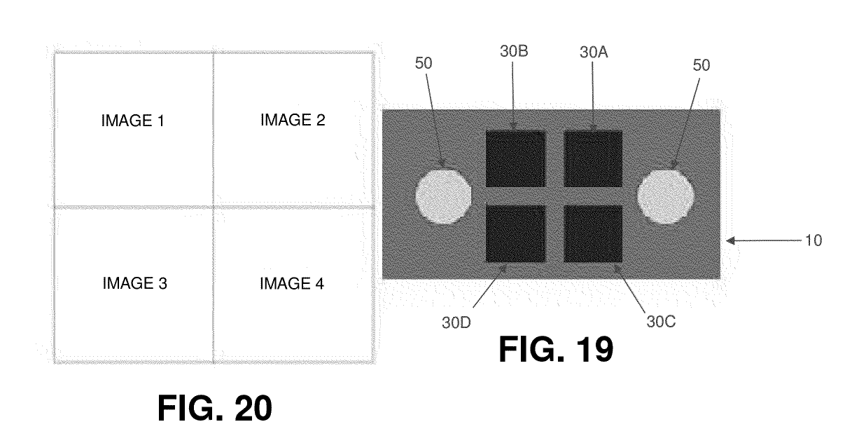

In another preferred form of the present invention, video input 30 may comprise a plurality of separate video inputs 30, each capable of acquiring visual images, whereby to gain additional views and/or perspective. By way of example but not limitation, and looking now at FIGS. 17-20, four separate video inputs 30A, 30B, 30C and 30D may be disposed on the tip of finger cot 10, preferably intermediate two illumination elements 50, so as to provide a multi-quadrant image. In this form of the invention, all four images (i.e., the images from video inputs 30A, 30B, 30C and 30D) may be displayed on a monitor at the same time (i.e., as a composite image, such as is shown in FIG. 20), or only one of the images may be displayed on the monitor at a given time, or several (but not all) of the images may be displayed on the monitor at the same time, etc.

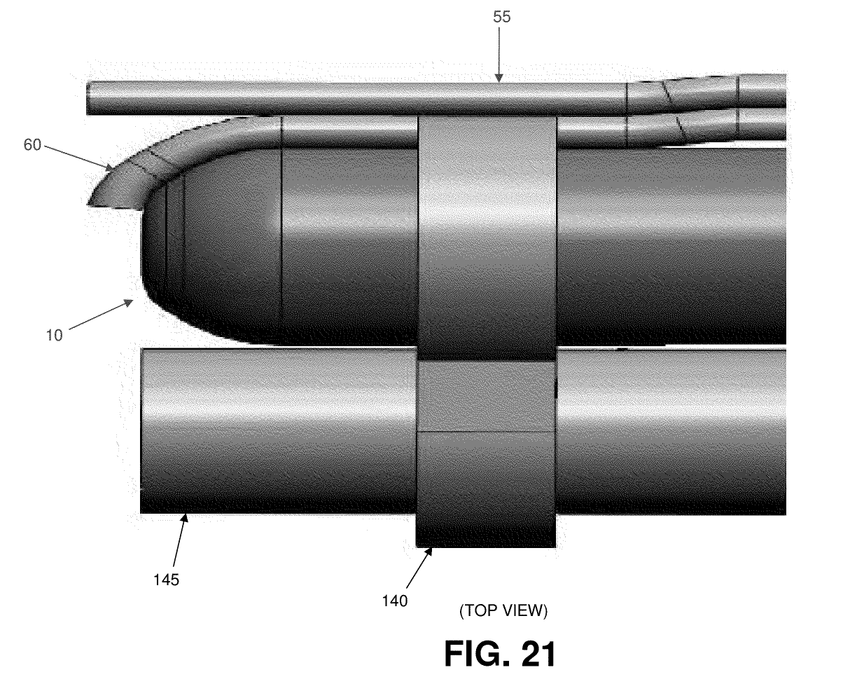

It is also possible for finger cot 10 to carry additional instrumentation. By way of example but not limitation, and looking now at FIG. 21, finger cot 10 may be provided with a mount 140 for slidably receiving an endoscope 145, whereby endoscope 145 can be telescoped relative to the finger of the user. This construction can be advantageous where it is necessary to provide visualization for tissue which is located at a site which cannot be accessed by the hand of the user, e.g., due to restricted access.

It should be appreciated that finger cot 10, auxiliary finger cot 130, and/or other means for mounting various elements (including video input 30, illumination elements 50, irrigation line 55, instrumentation 115, auxiliary devices 135, etc.) to the finger or fingers of a user are all preferably constructed so as to allow for positioning, rotation and re-positioning on the finger or fingers of a user, whereby to allow the user to determine how the elements are carried on the finger or fingers of the user.

### User Interface Controls

As noted above, hand-mounted, video-guided system 5 comprises a finger cot 10 for providing illumination, visualization and irrigation at the tip of a finger, and a fenestrated suction head 15 for providing suction at the base of the finger and/or at the palm of the hand. To this end, user interface controls are provided in connection with the illumination, visualization, irrigation and suction functions. By way of example but not limitation, user interface controls are provided to turn the illumination elements 50 on and off, to turn video input 30 on and off, to turn irrigation line 55 on and off, to turn suction line 90 on and off, etc. User interface controls may also be used to turn a video recorder on and off, to activate and/or telescope instrumentation relative to finger cot 10, to activate and/or telescope auxiliary instrumentation, to select one or several separate video inputs 30A, 30B, 30C, 30D, etc.

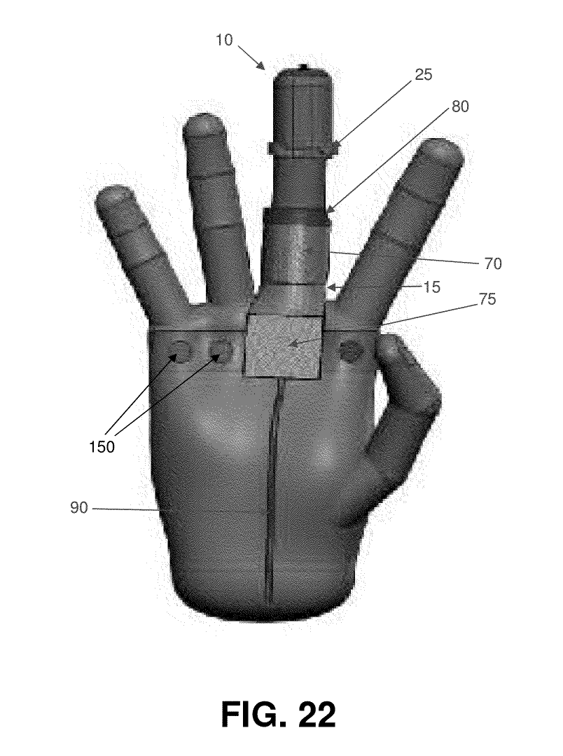

In one form of the invention, and looking now at FIG. 22, user interface controls are provided in the form of finger switches 150, preferably activated by the thumb of the user.

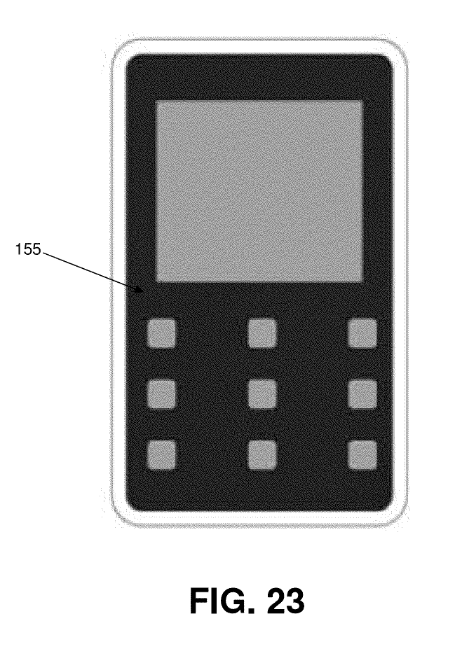

In another form of the invention, and looking now at FIG. 23, user interface controls are provided in the form of a touchscreen interface 155 (e.g., on a smartphone, a tablet computer, etc.).

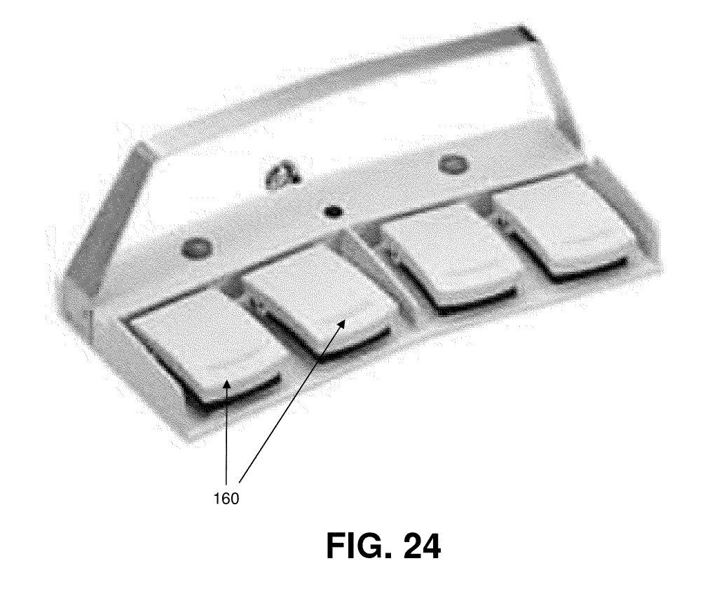

In still another form of the invention, and looking now at FIG. 24, user interface controls are provided in the form of foot switches 160.

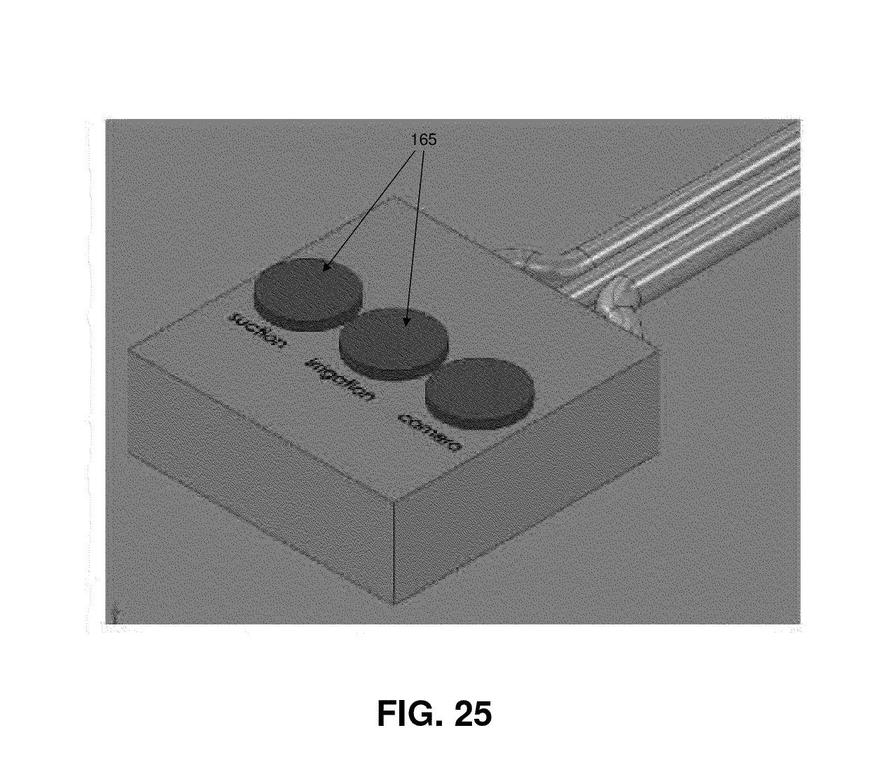

And in still another form of the invention, and looking now at FIG. 25, user interface controls are provided in the form of control box switches 165.

It should be appreciated that user interface controls may interface with the other elements of hand-mounted, video-guided system 5 via standard wiring (e.g., the video signal may be transmitted by USB cable, composite video cable, S-Video cable, etc.) or via wireless means (e.g., Bluetooth, Wi-Fi, etc.). Furthermore, if desired, hand-mounted, video-guided system 5 may interface with visualization apparatus (e.g., a video monitor) and/or other medical center elements (e.g., a central database, a telemedicine link where communications may be enabled to allow remote personnel to assist the user, etc.) via standard wiring or via wireless means (e.g., Bluetooth, Wi-Fi, etc.).

### Automatic Orientation of the Video

### Image Displayed to the User

If desired, hand-mounted, video-guided system 5 can include means for ensuring that the image projected on the video monitor is always presented “upright” to the user, regardless of the orientation of the user's hand (which carries hand-mounted, video-guided system 5). By way of example but not limitation, hand-mounted, video-guided system 5 can include accelerometers and/or gyroscopes for detecting the orientation of the hand of the user, or a “V” notch (or other visual cue) present on the image acquired by video input 30 to demarcate orientation, or manual switches to allow the user to “right” the image on the monitor, etc.

### Exemplary Constructions

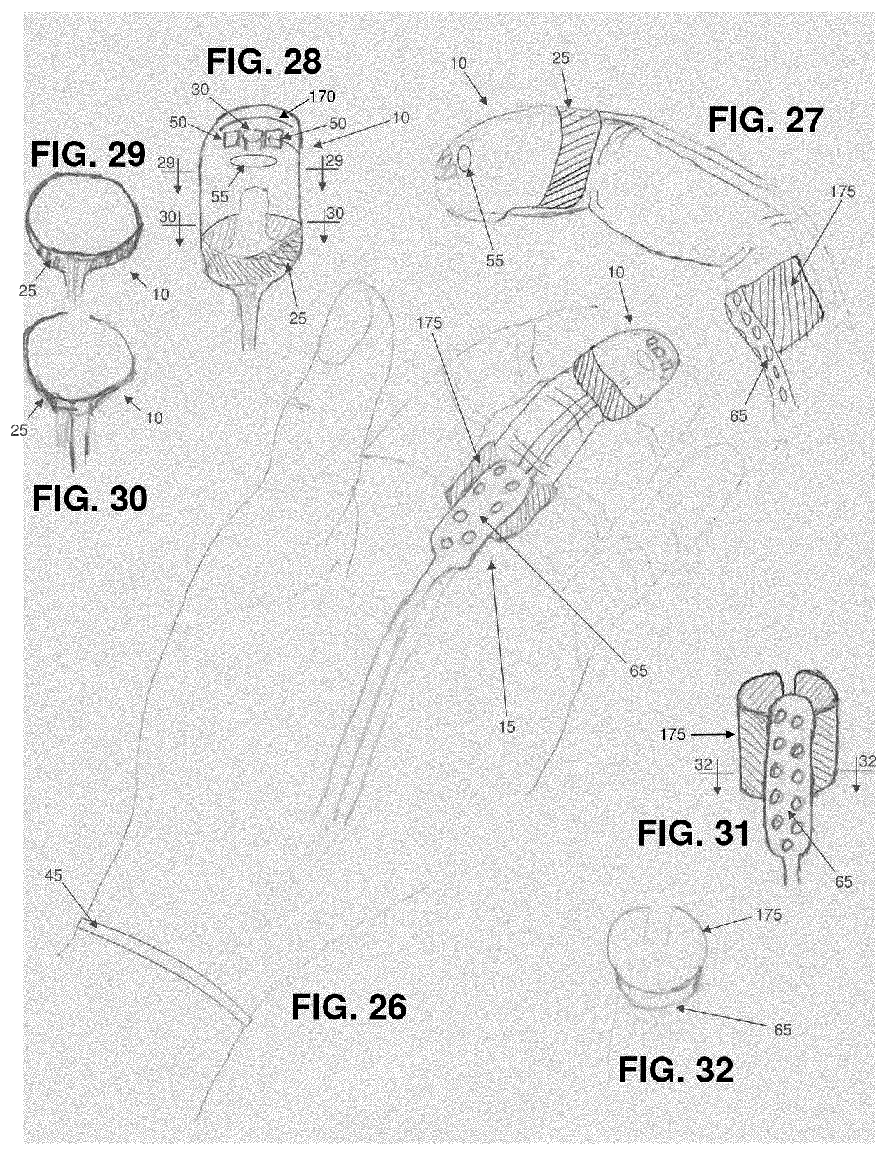

Looking next at FIGS. 26-32, there is shown an exemplary construction for hand-mounted, video-guided system 5. As seen in FIGS. 26-30, finger cot 10 is disposed on the distal phalanx of the finger, terminating short of the distalmost interphalangeal joint, with releasable locking ring 25 disposed distal to the distalmost interphalangeal joint. Or finger cot 10 may be longer than that shown. Video input 30 and illumination elements 50 are disposed on the distalmost tip of finger cot 10, adjacent to the mouth of irrigation line 55. A slit 170 may be formed in the distal tip of finger cot 10 so as to allow the finger of the user to be pushed distally out of the finger cot if desired in order to adjust the position of the video input 30 (and illumination elements 50 and irrigation line 55) on the finger. Fenestrated suction head 15 comprises an elongated hollow body 65 which seats against the underside of the user's finger at the base of the finger and extends against the palm of the hand at the base of the finger. As seen in FIGS. 26, 27, 31 and 32, a ring mount 175 (preferably bendable or malleable so as to be sizable) secures hollow body 65 to the finger of the user. Preferably ring mount 175 attaches to the proximal phalanx.

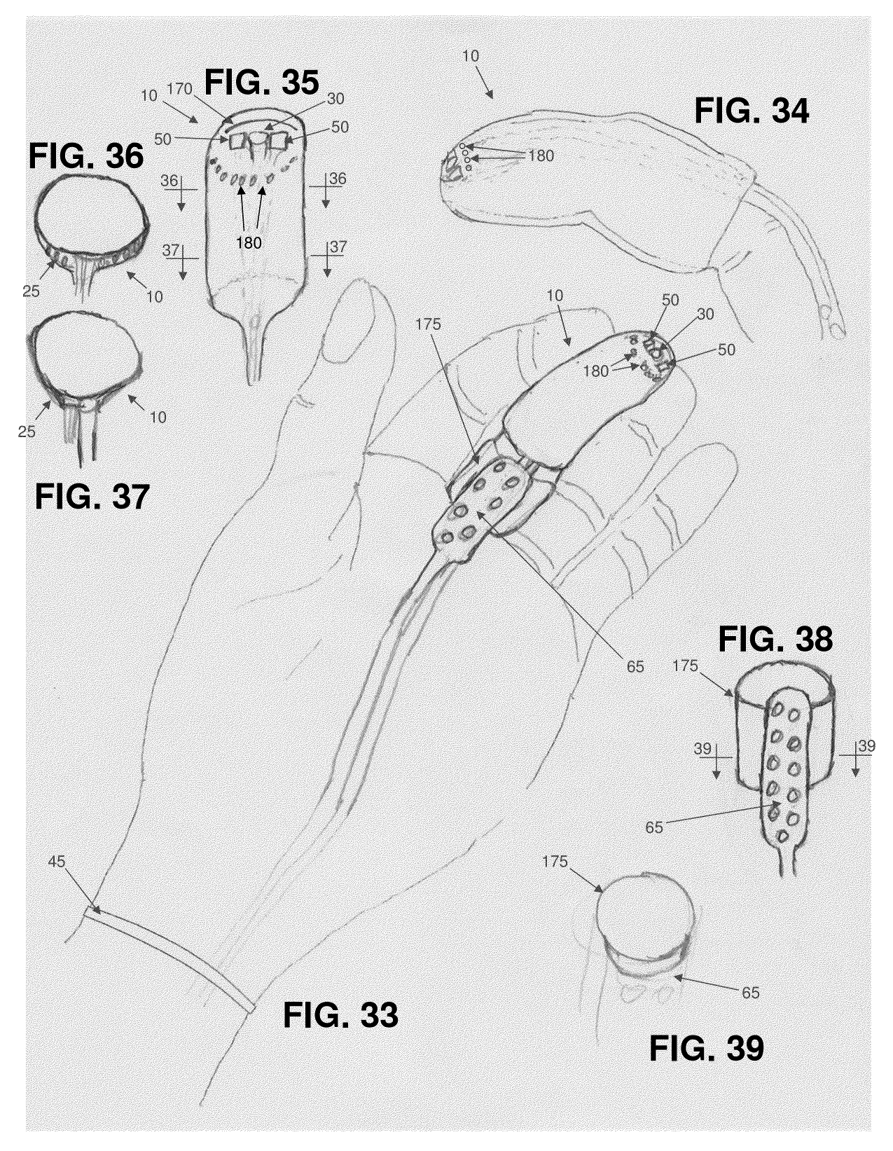

Looking next at FIGS. 33-39, there is shown another exemplary construction for hand-mounted, video-guided system 5. The hand-mounted, video-guided system 5 shown in FIGS. 33-39 is substantially the same as the hand-mounted, video-guided system 5 shown in FIGS. 26-32, except that (i) finger cot 10 extends along both the distal phalanx and the middle phalanx of the finger, terminating just short of the adjacent interphalangeal joint, (ii) releasable locking ring 25 may be omitted, and (iii) irrigation line 55 comprises a plurality of openings 180 at the distal tip of finger cot 10.

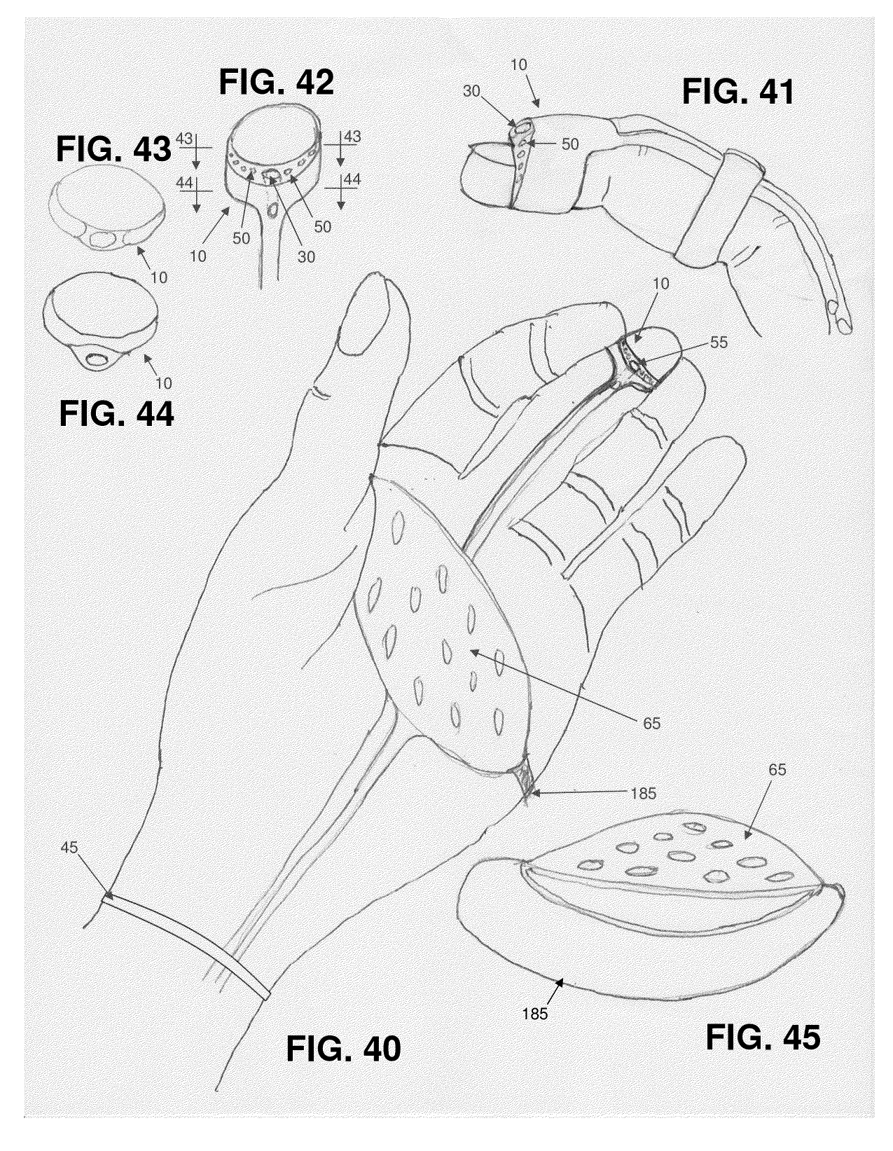

Looking next at FIGS. 40-45, there is shown another exemplary construction for hand-mounted, video-guided system 5. The hand-mounted, video-guided system 5 shown in FIGS. 40-45 is substantially the same as the hand-mounted, video-guided system 5 shown in FIGS. 26-32, except that (i) finger cot 10 terminates short of the distal tip of the finger of the user, and (ii) fenestrated suction head 15 comprises a hollow body 65 which seats against the palm of the hand and does not extend up the finger of the user. In this form of the invention, hollow body 65 may be mounted to the hand of the user via a retention strap 185.

### Additional Applications of the Hand-Mounted, Video-Guided System

As noted above, hand-mounted, video-guided system 5 comprises a video input 30, illumination elements 50, an irrigation line 55, and a fenestrated suction head 15. Hand-mounted, video-guided system 5 has particular application for treating diffuse bacterial peritonitis, inasmuch as it allows extensive lavage and video-guided decontamination. It also allows video- and palpation-guided finger dissection of loculated collections of infected material. Hand-mounted, video-guided system 5 also allows thorough evaluation of decontamination and hemostasis, without traction on tissues, in areas that would otherwise be difficult to visualize, particularly without applying traction.

It should be appreciated that hand-mounted, video-guided system 5 may be used for other applications as well. By way of example but not limitation, hand-mounted, video-guided system 5 may be advantageously used for:

(1) Large Pelvic Mass Resection: Where direct visualization of ureters and vascular structures is difficult, the system can be used to facilitate visualization of vascular structures, ureters, and ligamentous structures, substantially aiding in the safety and execution of the dissection;

(2) Large Soft Tissue Hematoma Evacuation: Through a small incision, the system allows a user to digitally fracture and visualize the evacuation of loculated collections of clotted blood, thereby ensuring thorough clot evacuation and satisfactory hemostasis (for example, with finger-mounted cautery or clips);

(3) Resection Of Distal Rectal Carcinoma: The system allows circumferential visualization and palpation of the most distal extent of the resection, allowing direct visualization of areas that cannot otherwise be seen—this allows improved visual confirmation of the adequacy of the resection;

(4) Adjunct To HALS Procedures, With The Device Worn On The Intra-abdominal Hand: The system provides improved visualization of ligamentous and vascular structures, which is of particular benefit for splenic and/or hepatic flexure mobilization;

(5) Trans-hiatal Esophagectomy (Orringer Procedure): The system allows for visual guidance along with manual dissection of the thoracic esophagus;

(6) Obtaining Satisfactory Hemostasis: At the conclusion of any procedure in a body cavity, in either the chest, abdomen/pelvis, or mediastinum, the system can be useful for both irrigation of the cavity to clear devitalized tissue, as well as to assess the adequacy of hemostasis and, additionally, visualize and apply cautery.

In addition to the foregoing, where hand-mounted, video-guided system 5 comprises additional instrumentation (e.g., cauterizing instrumentation, etc.), the system can be used to treat penetrating trauma. More particularly, the system allows for direct emergency wound evaluation, to assess the extent of injury and allow for immediate hemostasis and removal of foreign bodies. The system can also be used at the site of an accident by a trained EMT, or by a medic on site in military combat, augmented by real-time remote audio and video supervision by trauma surgeons, or in an emergency room setting by trained emergency room physicians or surgeons or other medical personnel.

Also, where hand-mounted, video-guided system 5 comprises a telescoping endoscope, with/without a mount for telescoping additional instrumentation, the system can be used for a routine GYN pelvic exam—the system allows for a simpler and more comfortable pelvic exam, by having video-guided digital examination, thereby allowing both visualization of the cervix and performance of the Pap smear, as well as palpation of the uterus and ovaries, and eliminating the need for an uncomfortable, often painful speculum examination. In this form of the invention, irrigation line 55 and/or fenestrated suction head 15 may be omitted.

Furthermore, the system may be used for pediatric neonatal intubation, which is a high-risk, difficult procedure—the system allows for simpler and potentially safer neonatal intubation, by allowing digital- and video-guided clearance of the airway and placement of an endotracheal tube, thereby eliminating the need for the cumbersome, potentially traumatic laryngoscopy. In this form of the invention, irrigation line 55 may be omitted or replaced with a suction line to remove mucous and meconium from the airway.

The system may also be used in dental applications.

The system may also be used for non-medical purposes. By way of example but not limitation, the system may be used to provide visualization in areas otherwise inaccessible during automotive or machinery production or repair, as well as uses in the construction industry.

### Further Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the present invention.

By way of example but not limitation, various elements of hand-mounted, video-guided system 5 may be omitted in certain circumstances.

Thus, for example, if desired, irrigation line 55, supplemental irrigation line 60 and fenestrated suction head 15 may all be omitted, in which case the system will essentially provide a digitally-mounted video input that allow video-guided imagery along with digital palpation.

Additionally, if desired, finger cot 10 may be incorporated in a larger element, e.g., a glove. In this case, auxiliary finger cot 130 may be incorporated in another portion of the glove, or other mounts may be incorporated in other portions of the glove for mounting instrumentation and auxiliary devices to the glove. Furthermore, where finger cot 10 is incorporated into a glove, fenestrated suction head 15 may also be integrated into the glove.

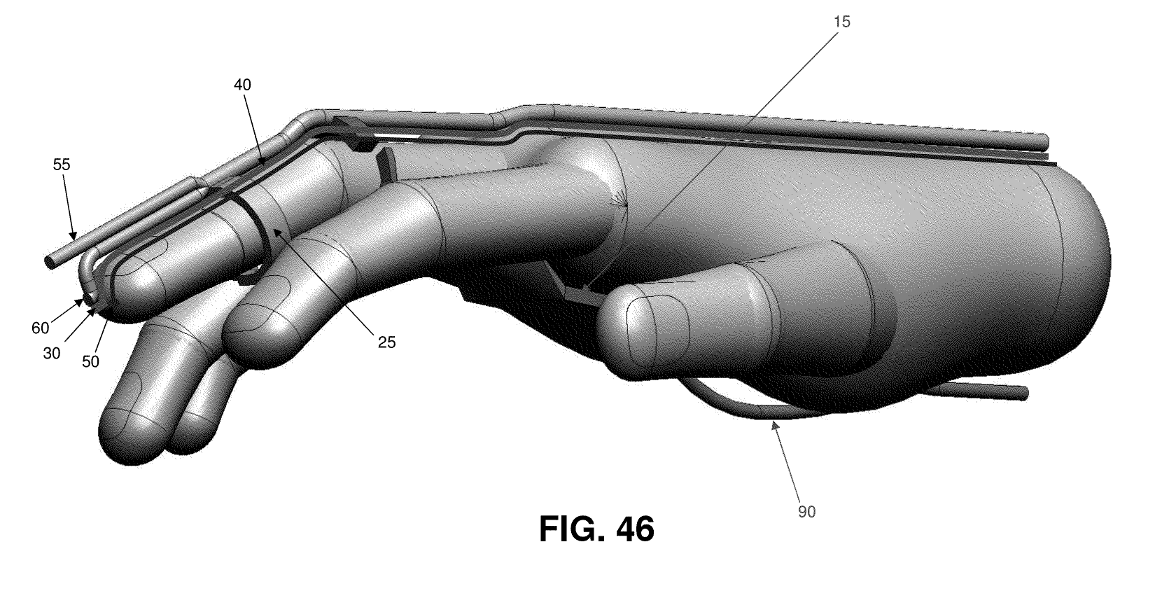

Furthermore, if desired, finger cot 10 may be omitted, with video input 30, illumination elements 50, irrigation line 55 and/or supplemental irrigation line 60 being mounted to the finger of a user via other means, e.g., a finger mount for securing video input 30, illumination elements 50, irrigation line 55 and/or supplemental irrigation line 60 to a finger of the user. By way of example but not limitation, and looking now at FIG. 46, video input 30, illumination elements 50, irrigation line 55 and/or supplemental irrigation line 60 may be carried directly by locking ring 25. Alternatively, the elements of the system may be carried by another type of finger mount, e.g., a “sizable” ring or clip (for example, a bendable or malleable ring or clip) or by other fixation devices compatible with the present invention.

Additionally, the system may provide more than one finger mount so that video input 30, illumination elements 50, irrigation line 55 and/or supplemental irrigation line 60 may be mounted to more than one finger of the user.

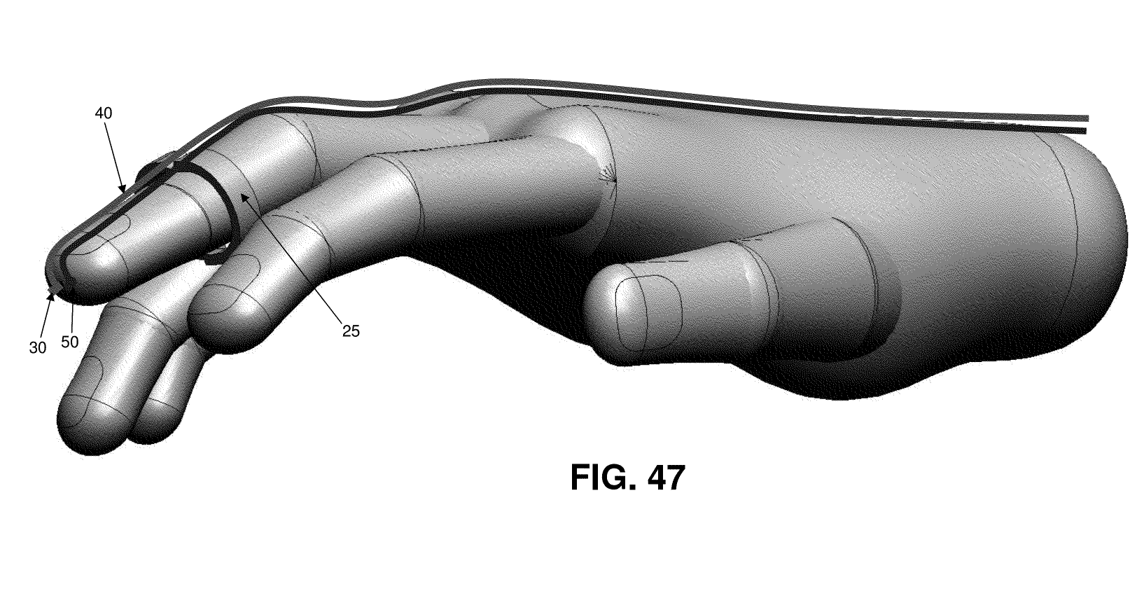

In one preferred form of the invention, and looking now at FIG. 47, the system comprises a locking ring 25 carrying video input 30 and illumination elements 50, whereby to provide a simple hand-mounted video system for use by the user.

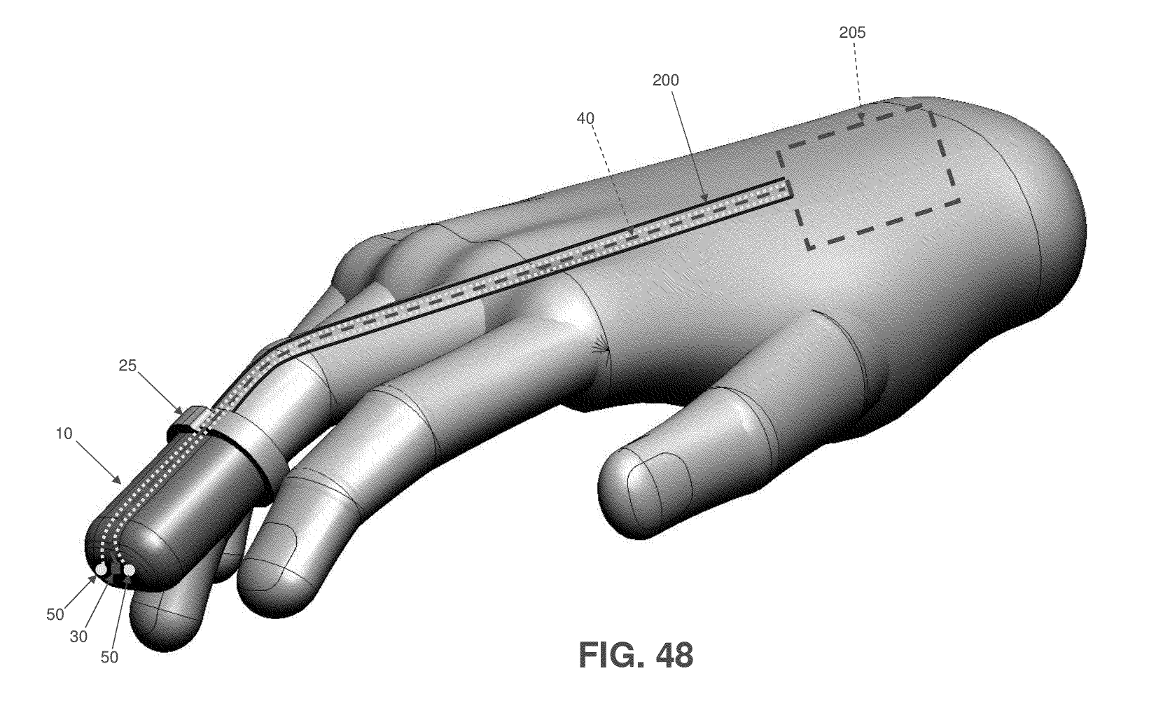

In another preferred embodiment, and looking now at FIG. 48, the system may comprise only the video function and accompanying light source (illumination elements), as a self-contained device, with the video input 30 and illumination elements 50 disposed at the fingertip, all preferably embedded in the finger cot 10 (or glove), with the transmission pathway 40 for video input 30 and the light fibers (or wires) for illumination elements 50 being encased in a cable 200 which is connected to a “box” 205 strapped to the user's wrist or forearm, wherein the box 205 contains video-processing apparatus (e.g., circuitry where video input 30 comprises a video chip, or a camera where video input 30 comprises a lens for a fiber optic transmission pathway 40), means for energizing illumination elements 50 (e.g., a battery where illumination elements 50 comprise an LED, or a light source where illumination elements comprise a light fiber), a Bluetooth (or Wi-Fi) transceiver for enabling the system to communicate with a remote system (e.g., a video display, a computer, a medical center database, a telemedicine site, etc.), and a rechargeable battery for powering the system components. This construction can be particularly useful for a reusable GYN device for pelvic examinations and PAP testing, worn under a single-use, disposable glove, that would be specifically designed to provide a clear membrane “window” positioned over the lens of video input 30.

These and other changes of the sort may be made while remaining within the principles and scope of the present invention.

What is claimed is:

1. A hand-mounted, video-guided system comprising:
an element for mounting to at least one finger of a user;
a video input carried by said element;
an irrigation line carried by said element;
a fenestrated suction head for mounting to the hand of a user, said fenestrated suction head comprising a hollow body having fenestrations formed therein, and a suction line having a fenestrated distal end disposed within the interior of said hollow body.

2. A hand-mounted, video-guided system according to claim 1 wherein said element comprises a finger cot.

3. A hand-mounted, video-guided system according to claim 2 wherein said finger cot comprises a portion of a glove.

4. A hand-mounted, video-guided system according to claim 1 wherein the element comprises a ring.

5. A hand-mounted, video-guided system according to claim 4 wherein said ring comprises a sizable ring.

6. A hand-mounted, video-guided system according to claim 1 wherein the tactile sense of the user is maintained when said element is mounted to said at least one finger.

7. A hand-mounted, video-guided system according to claim 2 wherein said finger cot mounts to one finger of a user.

8. A hand-mounted, video-guided system according to claim 2 wherein said finger cot mounts to more than one finger of a user.

9. A hand-mounted, video-guided system according to claim 2 further comprising a ring for securing said finger cot to at least one finger of a user.

10. A hand-mounted, video-guided system according to claim 9 wherein said ring comprises a releasable locking ring.

11. A hand-mounted, video-guided system according to claim 2 wherein said video input is formed integral with said finger cot.

12. A hand-mounted, video-guided system according to claim 2 wherein said video input is mounted to said finger cot.

13. A hand-mounted, video-guided system according to claim 2 wherein the image captured by said video input is transferred to display apparatus via a transmission pathway.

14. A hand-mounted, video-guided system according to claim 13 wherein said video input comprises a video chip and said transmission pathway comprises a wire.

15. A hand-mounted, video-guided system according to claim 13 wherein said video input comprises at least one lens and said transmission pathway comprises a fiberoptic cable.

16. A hand-mounted, video-guided system according to claim 2 wherein said video input is telescopically carried by said finger cot.

17. A hand-mounted, video-guided system according to claim 2 wherein said irrigation line is formed integral with said finger cot.

18. A hand-mounted, video-guided system according to claim 2 wherein said irrigation line is mounted to said finger cot.

19. A hand-mounted, video-guided system according to claim 2 wherein said irrigation line is telescopically carried by said finger cot.

20. A hand-mounted, video-guided system according to claim 2 further comprising at least one illumination element for illuminating the region which is monitored by said video input.

21. A hand-mounted, video-guided system according to claim 20 wherein said at least one illumination element comprises at least one LED.

22. A hand-mounted, video-guided system according to claim 20 wherein said at least one illumination element is telescopically carried by said finger cot.

23. A hand-mounted, video-guided system according to claim 20 wherein said at least one illumination element comprises a light fiber.

24. A hand-mounted, video-guided system according to claim 20 further comprising a supplemental irrigation line for irrigating said video input.

25. A hand-mounted, video-guided system according to claim 20 further comprising a mount for supporting instrumentation to the at least one finger of a user.

26. A hand-mounted, video-guided system according to claim 25 wherein said mount telescopically supports instrumentation to at least one finger of a user.

27. A hand-mounted, video-guided system according to claim 25 wherein said mount is carried by said finger cot.

28. A hand-mounted, video-guided system according to claim 25 wherein said mount is independent of said finger cot.

29. A hand-mounted, video-guided system according to claim 28 wherein said finger cot is mounted to a finger and said mount is mounted to another finger.

30. A hand-mounted, video-guided system according to claim 25 wherein said instrumentation is selected from the group consisting of a biopsy instrument, a cauterizing device, a suction device, a laser, an ultrasound imager, an ablative device, a Blue light, a fluorescent light, an endoscope, a balloon, and a device for deployment of glue.

31. A hand-mounted, video-guided system according to claim 2 further comprising a cuff for securing said irrigation line to the wrist of a user.

32. A hand-mounted, video-guided system according to claim 2 further comprising a blunt hood for protecting said video input.

33. A hand-mounted, video-guided system according to claim 2 further comprising an auxiliary finger cot for mounting to at least one other finger of a user, the auxiliary finger cot carrying instrumentation.

34. A hand-mounted, video-guided system according to claim 2 wherein said fenestrated suction head is mounted to at least one finger of a user.

35. A hand-mounted, video-guided system according to claim 2 wherein said fenestrated suction head is mounted to at least one finger of a user by a ring.

36. A hand-mounted, video-guided system according to claim 35 wherein said irrigation line is mounted to said ring.

37. A hand-mounted, video-guided system according to claim 36 wherein said irrigation line is movably mounted to said ring.

38. A hand-mounted, video-guided system according to claim 37 wherein said fenestrated suction head is mounted to the palm of the hand of a user.

39. A hand-mounted, video-guided system according to claim 38 wherein said fenestrated suction head is mounted to the palm of the hand of a user by a strap.

40. A hand-mounted, video-guided system according to claim 39 wherein said irrigation line is mounted to said strap.

41. A hand-mounted, video-guided system according to claim 39 wherein said irrigation line is movably mounted to said strap.

42. A hand-mounted, video-guided system according to claim 2 wherein said fenestrated suction head is mounted to at least one finger and the palm of the hand of a user.

43. A hand-mounted, video-guided system according to claim 2 wherein said video input and said irrigation line are carried by said finger cot so as to minimize interference with the tactile sensation of the fingers of a user.

44. A hand-mounted, video-guided system according to claim 2 wherein said video input and said irrigation line are carried by said finger cot so that they are clear of the pad of at least one finger of a user.

45. A hand-mounted, video-guided system according to claim 2 wherein said video input and said irrigation line are carried by said finger cot at said distal tip of said finger cot.

46. A hand-mounted, video-guided system according to claim 2 wherein said video input and said irrigation line are carried by said finger cot along at least one side of the at least one finger of a user.

47. A method for treating a patient, comprising:
providing a hand-mounted, video-guided system comprising:
an element for mounting to at least one finger of a user;
a video input carried by said element;
an irrigation line carried by said element;
a fenestrated suction head for mounting to the hand of a user, said fenestrated suction head comprising a hollow body having fenestrations formed therein, and a suction line having a fenestrated distal end disposed within the interior of said hollow body;
mounting said apparatus to the hand of a user;
advancing the hand of a user into a body cavity; and
providing lavage therapy to the patient using said irrigation line and said fenestrated suction head while visualizing the anatomy using said video input.

48. A method according to claim 47 wherein said element comprises a finger cot.

49. A method according to claim 48 wherein said finger cot comprises a portion of a glove.

50. A method according to claim 47 wherein the element comprises a ring.

51. A method according to claim 50 wherein said ring comprises a sizable ring.

52. Apparatus comprising:
an element for mounting to at least one finger of a user; and
a video input carried by said element.

53. Apparatus according to claim 52 wherein said element comprises a finger cot.

54. Apparatus according to claim 53 wherein said finger cot comprises a portion of a glove.

55. Apparatus according to claim 52 wherein the element comprises a ring.

56. Apparatus according to claim 55 wherein said ring comprises a sizable ring.

57. Apparatus according to claim 52 further comprising a suction line carried by said element.

58. A method for examining or treating a patient, comprising:
providing apparatus comprising:
an element for mounting to at least one finger of a user; and
a video input carried by said element
mounting said apparatus to the hand of a user;
advancing the hand of a user into a body opening or cavity; and
visualizing the anatomy using said video input.

59. A method according to claim 58 wherein the apparatus further comprises a suction line carried by said element, and wherein said method further comprises providing suction to the body opening or cavity while simultaneously visualizing and palpating the anatomy and pathology.

60. A method for viewing a confined space and/or performing a task in the confined space, comprising:
providing apparatus comprising:
an element for mounting to at least one finger of a user; and
a video input carried by said element;
mounting said apparatus to the hand of a user;
advancing the hand of a user into a confined space; and
viewing the confined space using the video input.

61. A method according to claim 60, further comprising performing a task in the confined space while viewing the confined space using the video input.

62. A method according to claim 61 wherein the task comprises providing medical therapy.

63. A method according to claim 61 wherein the task comprises the assembly or disassembly of a mechanical or electrical device.

64. A hand-mounted video system comprising:
a video input for acquiring video data; and
means for mounting said video input to the hand of a user.
